(19) United States
(12) Patent Application Publication
(10) Pub. No.: US 2004/0153018 A1
(43) Pub. Date: Aug. 5, 2004

(54) Shave patches

(76) Inventor: Symeon Brown, Crete, IL (US)

Correspondence Address: Symeon Brown, 3654 Ronald Road, Crete, IL, 60417, US

(21) Appl. No.: 10/357,188

(22) Filed: Feb. 3, 2003

(51) Int. Cl. A61F 13/00

(52) U.S. Cl. 602/41

(57) ABSTRACT

The “SHAVE PATCHES” will take the place of bathroom tissue that men use after cutting themselves while shaving. It's simply a thin piece of papertowel like material that is moistened with an antibacterial solution which delivers a cool and refreshing sensation to the sensitive cut area on the face. The strips measure 3 to 4 centimeters wide and are rolled in the form of a dental floss wheel. The tip protrudes from the opening of the dispenser allowing an individual to tear off a pre-cut piece to apply to the affected area.

### BACKGROUND OF THE INVENTION

This product is the first of it's kind and this is design for men. This product will be apart of men's cosmetic.

### BRIEF SUMMARY OF THE INVENTION

The shave patches will take the place of toilet tissue that men use after shaving when a cut accurse

### SPECIFICATION

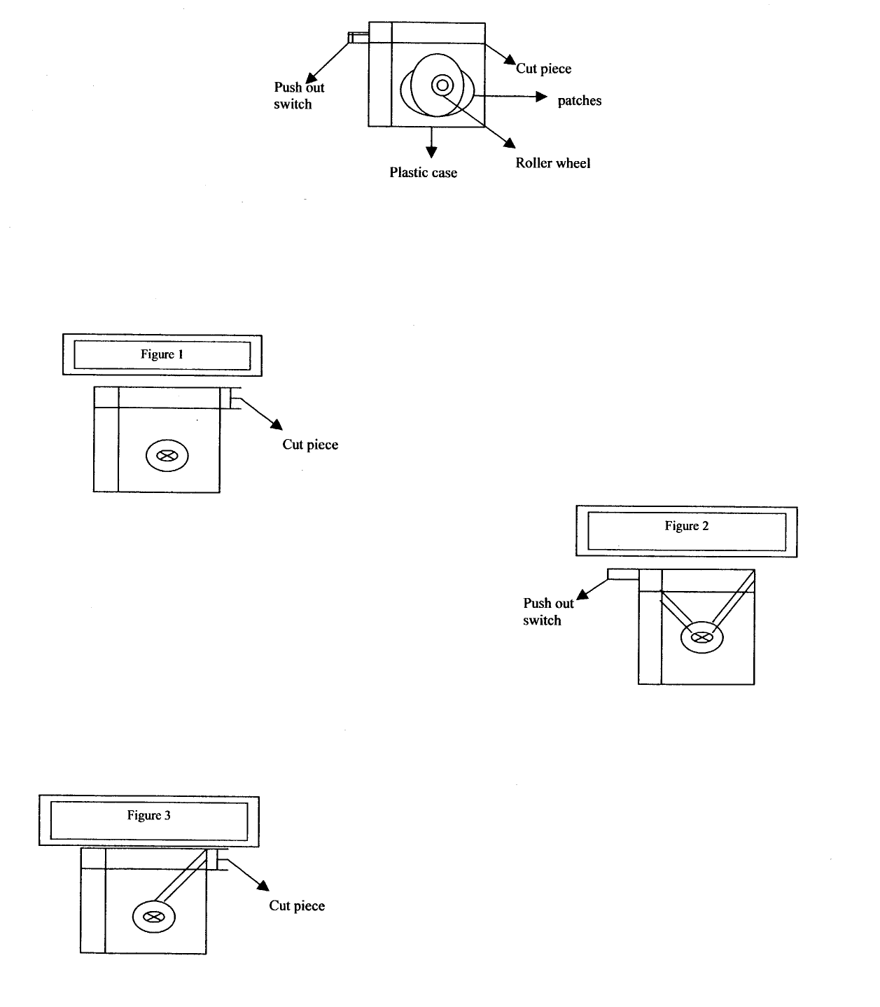

Shave patches is an idea I came up with when I was shaving. So many times men and women shave and have the misfortune of cutting themselves or getting a small nick. That's when I came up with SHAVE PATCHES. This invention I developed will take the place of toilet tissue used on your face forever. So many times when a man cuts himself shaving he goes and grabs a tiny piece of toilet tissue and puts that piece on the cut on his face, and waits awhile until the blood dries up. The Shave Patches will take the place of the toilet tissue. I took a sheet of paper towel and I cut it approximately 3 or 4 cm wide and about 12 inches long. I then took a roller off a dental floss box. Took the paper towel, saturated with alcohol took it out and let it sit for a few hours until dry. I then wrapped it around a roller and I snapped the plastic cover back into place with the tip of the paper towel protruding from the top. Next I put a cut piece on the mouth of the dispenser (see FIG. 1) So when you push the switch (see FIG. 2) the patches will release smooth and easy, right out of the mouth of the dispenser. Once you release the switch it will cut the patch (see FIG. 3) right into your finger tips. You put the dot size patch onto the cut on the face for a smooth, dry, sensational feeling.

1. What I claim as my invention is that the shave patches will stick to your face replacing pieces of toilet tissue that you stick to your face after you get a cut shaving your face.

2. I claim that it will also give your face a sting (sensational feeling) after you stick the patch on your fresh cut.

3. I also claim it will dry out your blood spot in minutes
